(19) United States
(12) Patent Application Publication
(10) Pub. No.: US 2023/0157606 A1
(43) Pub. Date: May 25, 2023

(54) PHYSICAL STRENGTH EVALUATION SYSTEM, PHYSICAL STRENGTH EVALUATION METHOD, AND COMPUTER PROGRAM

(71) Applicants: ASICS CORPORATION, Kobe-shi (JP); CASIO COMPUTER CO., LTD., Tokyo (JP)

(72) Inventors: Ken Kusano, Kobe-shi (JP); Noriko Nishimura, Kobe-shi (JP); Masaru Ichikawa, Kobe-shi (JP); Takehiro Tagawa, Kobe-shi (JP); Shosaku Suzuki, Tokyo (JP); Takeshi Yokoi, Tokyo (JP)

(73) Assignees: ASICS CORPORATION, Kobe-shi (JP); CASIO COMPUTER CO., LTD., Tokyo (JP)

(21) Appl. No.: 18/055,742

(22) Filed: Nov. 15, 2022

(30) Foreign Application Priority Data
Nov. 25, 2021 (JP) 2021-191556

(51) Int. Cl. A61B 5/22; A61B 5/0205; A63B 24/00

(52) U.S. Cl. CPC A61B 5/224; A61B 5/0205; A63B 24/0062; A61B 5/02438

(57) ABSTRACT

In a physical strength evaluation system, a reference storage stores a walking heart rate reference in which a correspondence relationship of a standard value of a relative heart rate with respect to a walking speed is defined for every gender and every age, a speed acquisition unit acquires a measured value of a walking speed of a subject, a heart rate acquisition unit acquires a measured value of the relative heart rate of the subject. A standard value derivation unit derives the standard value of the relative heart rate corresponding to the measured value of the walking speed that has been acquired, based on the walking heart rate reference. A level determination unit determines an evaluation value of a physical strength level of the subject, based on a difference between the measured value of the relative heart rate and the standard value of the relative heart rate.

### BACKGROUND OF THE INVENTION

### 1. Field of the Invention

The present invention relates to a physical strength evaluation system. In particular, the present invention relates to a system for analyzing a physical condition of a walker, based on a heart rate.

### 2. Description of the Related Art

In recent years, the heart rate during exercise or in everyday life can be readily measured by use of a heart rate belt worn around the chest or a heart rate sensor built in a wrist watch. A technique is known for estimating a reference heart rate that serves as an anaerobic threshold from a running speed during running at predetermined exercise intensity (see, for example, JP 6063743 B2). In this technique, the reference heart rate is estimated, based on a relationship that the amount of change in the heart rate with respect to the amount of change in the running speed is constant.

Here, various studies have been conducted on the relationship between the amount of change in the heart rate with respect to the amount of change in the running speed during running. However, the above relationship is not always applied to the relationship of the heart rate with respect to the walking speed during so-called walking. In addition, it is considered that the tendency of the heart rate varies depending on a difference in age or gender. Hence, the above relationship is not applicable to evaluations of exercise of walkers of various ages and genders.

### SUMMARY OF THE INVENTION

The present invention has been made in view of such circumstances, and has an object to provide a technique capable of measuring a walking speed and a heart rate to evaluate a physical strength in walking exercise.

In order to address the above issues, a physical strength evaluation system according to an aspect of the present invention includes: a reference storage structured to store a walking heart rate reference in which a correspondence relationship of a standard value of a relative heart rate with respect to a walking speed is defined; a speed acquisition unit structured to acquire a measured value of a walking speed of a subject; a heart rate acquisition unit structured to acquire a measured value of a relative heart rate of the subject; a standard value derivation unit structured to derive the standard value of the relative heart rate corresponding to the measured value of the walking speed that has been acquired, based on the walking heart rate reference; a level determination unit structured to determine an evaluation value of a physical strength level of the subject, based on a difference between the measured value of the relative heart rate and the standard value of the relative heart rate; and an output unit structured to output the evaluation value of the physical strength level that has been determined.

Here, the “relative heart rate” may be a ratio of the measured value of the heart rate to the maximum heart rate. The “standard value of the relative heart rate” may be an average value of the relative heart rates that have been measured for a plurality of subjects. The “difference between the measured value of the relative heart rate and the standard value of the relative heart rate” may be a numerical difference or a level difference, or may be a magnitude of a ratio of the measured value to the standard value. The “physical strength evaluation system” may include only a single information terminal, may include a combination of an information terminal and a measurement device, or may include a combination of an information terminal and a server. Alternatively, the “physical strength evaluation system” may include a combination of an information terminal, a measurement device, and a server, may include a combination of a measurement device and a server, or may include only a server. According to this aspect, the physical strength level of the subject can be evaluated in a simple method in which the physical strength level is relatively high in a case where the relative heart rate of the subject is lower than the standard value, and the physical strength level is relatively low in a case where the relative heart rate of the subject is higher than the standard value.

### BRIEF DESCRIPTION OF THE DRAWINGS

FIG. 1 is a diagram illustrating a configuration of a physical strength evaluation system;

FIG. 2 is a functional block diagram illustrating respective configurations of the physical strength evaluation system;

FIG. 3 is a diagram illustrating a walking heart rate reference in which a correspondence relationship of a standard value of a relative heart rate with respect to a walking speed is defined;

FIG. 4 is a diagram illustrating a relationship between a difference between a standard value and a measured value of the relative heart rate, and a heart rate score;

FIGS. 5A and 5B are diagrams illustrating walking heart rate references for every gender and every age group;

FIG. 6 is a diagram schematically illustrating a screen example for registering attributes of a subject;

FIG. 7 is a diagram schematically illustrating a screen example of displaying a physical strength level;

FIG. 8 is a functional block diagram illustrating respective configurations of a physical strength evaluation system in a second embodiment;

FIG. 9 is a diagram illustrating a running heart rate reference in which a correspondence relationship of a standard value of a relative heart rate with respect to a running speed is defined; and

FIGS. 10A and 10B are diagrams illustrating running heart rate references for every gender and every age group.

### DETAILED DESCRIPTION OF THE INVENTION

The invention will now be described by reference to the preferred embodiments. This does not intend to limit the scope of the present invention, but to exemplify the invention.

Hereinafter, the present invention will be described based on preferred embodiments with reference to the drawings. In embodiments and modifications, the same or equivalent components are denoted by the same reference numerals, and overlapping descriptions will be omitted as appropriate.

### First Embodiment

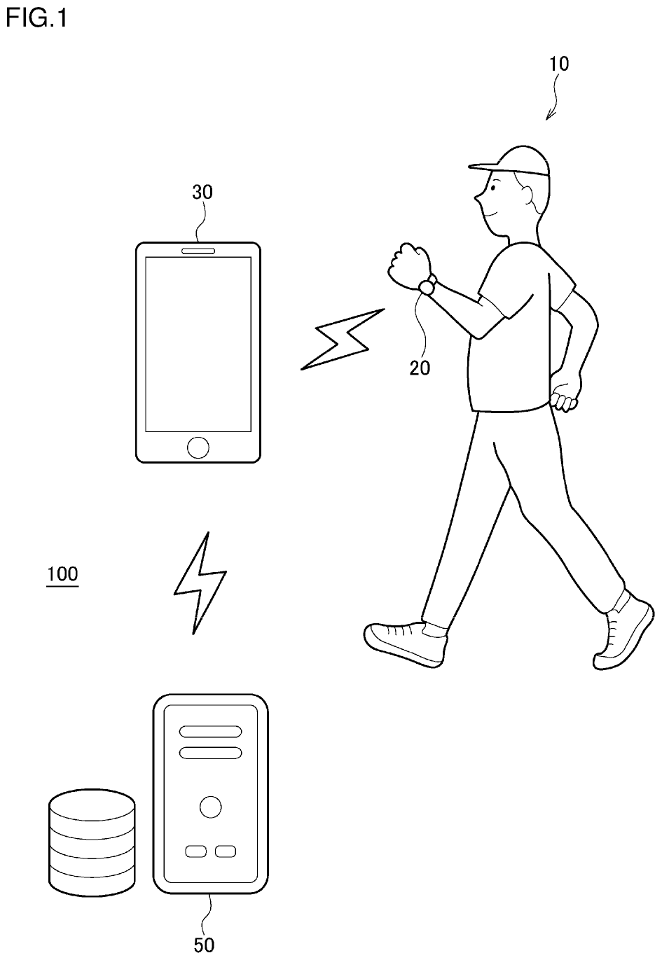

FIG. 1 is a diagram illustrating a configuration of a physical strength evaluation system 100. The physical strength evaluation system 100 includes at least an information terminal 30, and is synchronized through communication with a measurement device 20, which is worn on an arm of a subject 10 doing walking exercise, and which measures a heart rate and the like, or with an information management server 50. The measurement device 20 is, for example, a sports watch or a smart watch capable of acquiring location information and a heart rate. Note that the measurement device 20 is not limited to a wrist watch-type device, and may be a belt-type heart rate meter capable of acquiring the heart rate by being wound around the chest or a wrist of the subject. Note that the type of the device is not limited to the belt-type heart rate meter, as long as the walking log data to be described later is acquirable.

The subject 10 does walking exercise by wearing, on its arm, the measurement device 20 capable of measuring values such as a walking time, a walking distance, a walking speed, the number of steps, a step length, and a heart rate, and recording the values as a log (hereinafter, also referred to as a “walking log”) of the walking exercise. The subject 10 operates a button of the measurement device 20 at the start of walking to start measurements and start recording a walking log. While the walking exercise is being done, the measurement device 20 measures an elapsed time from the start of recording as the walking time, and measures the walking distance, based on the location information. The measurement device 20 measures the walking speed based on the walking time and the walking distance. The measurement device 20 measures, by a built-in optical heart rate sensor, the heart rate based on a change in blood flow volume on a skin surface of the wrist. After the walking exercise and recording of the walking log end, the measurement device 20 transmits the walking log data such as the walking time, the walking distance, the walking speed, the number of steps, the step length, and the heart rate to the information terminal 30 through wireless communication.

The information terminal 30 is a portable terminal such as a smartphone carried by the subject 10. The information terminal 30 evaluates a physical strength level of the subject 10 based on the walking log data such as the walking time, the walking distance, the walking speed, the number of steps, the step length, and the heart rate that have been received from the measurement device 20. The information terminal 30 transmits the walking log data such as the walking time, the walking distance, the walking speed, the number of steps, the step length, and the heart rate of the subject 10 to the information management server 50 through communication. The information terminal 30 may be a tablet terminal or a personal computer possessed by the subject.

Note that the information terminal 30 may acquire not only the walking log from the measurement device 20 but also the walking log by use of various sensors or communication functions built in the information terminal 30 itself. In such a case, the information terminal 30 may acquire only the heart rate from the measurement device 20 such as the belt-type heart rate meter, and may acquire and record the other walking logs in the information terminal 30.

The information management server 50 is a server computer that is connected to the Internet, and transmits and receives data to and from a plurality of information terminals 30. The information management server 50 acquires and accumulates data such as the walking time, the walking distance, the walking speed, the number of steps, the step length, and the heart rate, as the walking log data of the subject 10 that have been received from the information terminal 30. The information management server 50 transmits the accumulated walking log data to the information terminal 30 in response to a request from the information terminal 30.

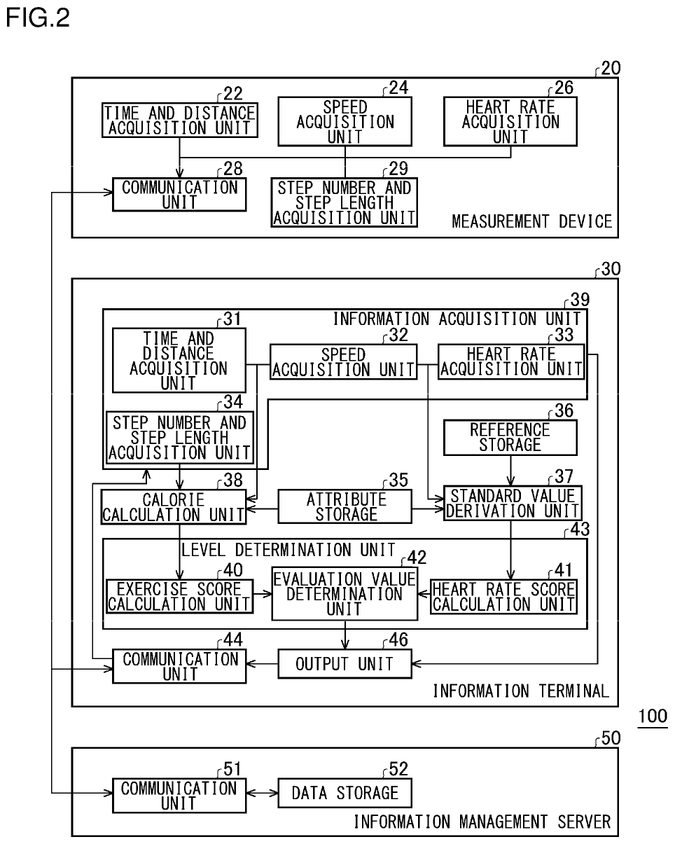

FIG. 2 is a functional block diagram illustrating respective configurations of the physical strength evaluation system 100. The physical strength evaluation system 100 according to the present embodiment includes at least a hardware configuration and a software configuration included in the information terminal 30. However, at least one of the measurement device 20 and the information management server 50 may be included as a part of the physical strength evaluation system 100. Thus, various combinations of configurations are conceivable for the physical strength evaluation system 100. For example, the physical strength evaluation system 100 may include only the information terminal 30, may include a combination of the information terminal 30 and the measurement device 20, or may include a combination of the information terminal 30 and the information management server 50. Alternatively, the physical strength evaluation system 100 may include a combination of the information terminal 30, the measurement device 20, and the information management server 50, may include a combination of the measurement device 20 and the information management server 50, or may include only the information management server 50. In FIG. 2, with regard to the measurement device 20, the information terminal 30, and the information management server 50, functional blocks implemented by cooperation of various hardware configurations and software configurations are illustrated. Therefore, it is to be understood by those skilled in the art that these functional blocks can be implemented in various forms of only hardware, only software, or a combination of them. Hereinafter, functions of the measurement device 20, the information terminal 30, and the information management server 50 will be described.

The measurement device 20 includes a time and distance acquisition unit 22, a speed acquisition unit 24, a heart rate acquisition unit 26, a communication unit 28, and a step number and step length acquisition unit 29.

The time and distance acquisition unit 22 measures a walking time from a walking start time, that is, a measurement start time, based on time information counted by a timer. In addition, the time and distance acquisition unit 22 measures a walking distance, based on the location information of a current location received by a global positioning system (GPS) module from a satellite positioning system and information of acquired date and time.

The speed acquisition unit 24 calculates a walking speed, based on the information of the walking time and the walking distance that have been acquired by the time and distance acquisition unit 22, and acquires its average value as a measured value of the walking speed of the subject 10.

The heart rate acquisition unit 26 acquires an average heart rate per unit time of the subject 10, based on measured results of the heart rate sensor. Note that in the present embodiment, as will be described later, the information terminal 30 calculates a relative heart rate, based on the measured value of the heart rate of the subject 10 and the maximum heart rate. However, in a modification, the heart rate acquisition unit 26 may calculate the relative heart rate in the measurement device 20.

The step number and step length acquisition unit 29 measures, by a motion sensor, the number of steps of the subject during walking, and calculates an average value of the step lengths, based on the number of steps and the walking distance.

The measured values that have been acquired by the time and distance acquisition unit 22, the speed acquisition unit 24, the heart rate acquisition unit 26, and the step number and step length acquisition unit 29 are transmitted to the information terminal 30 via the communication unit 28, after the walking exercise and the measurements end.

The information terminal 30 includes an information acquisition unit 39, an attribute storage 35, a reference storage 36, a standard value derivation unit 37, a calorie calculation unit 38, a level determination unit 43, a communication unit 44, and an output unit 46. The information acquisition unit 39 includes a time and distance acquisition unit 31, a speed acquisition unit 32, a heart rate acquisition unit 33, and a step number and step length acquisition unit 34. The level determination unit 43 includes an exercise score calculation unit 40, a heart rate score calculation unit 41, and an evaluation value determination unit 42.

The information acquisition unit 39 acquires data of the walking time, the walking distance, the walking speed, the heart rate, the number of steps, and the step length from the measurement device 20 via the communication unit 44. The attribute storage 35 stores user information and attribute information of the subject 10, which have been registered beforehand. The attribute information of the subject 10 includes, for example, information of gender, age, weight, and the like.

The calorie calculation unit 38 calculates calorie consumption of walking per minute, based on the information of the walking speed, the weight, and the like, and also calculates an integrated value of the calorie consumption of the entire walking exercise. Note that, in order to eliminate the influence of a case where the walking time is too long, the calorie calculation unit 38 calculates, for example, the calorie consumption up to the walking time of 30 minutes at the maximum.

The exercise score calculation unit 40 calculates an exercise score based on the calorie consumption. The exercise score calculation unit 40 stores a calculation formula for calculating an exercise score for every gender and every age, and calculates an exercise score in a calculation formula corresponding to the gender and the age of the subject. Only in a case where the walking time, the number of steps, and the walking distance in walking exercise respectively satisfy predetermined conditions, the exercise score calculation unit 40 calculates the exercise score of such walking exercise. For example, as the predetermined conditions, in a case where in walking exercise, the walking time is 10 minutes or more, the number of steps is 600 steps or more, and the walking distance is 300 m or more, the exercise score calculation unit 40 calculates an exercise score, based on calorie consumption in such walking exercise and a calculation formula corresponding to the gender and age of the subject.

The reference storage 36 stores a walking heart rate reference in which a correspondence relationship of a standard value of the relative heart rate with respect to the walking speed is defined. The standard value of the relative heart rate may be an average value of the relative heart rates that have been collected beforehand as samples for every walking speed. The walking heart rate reference denotes a regression equation having the walking speed as an explanatory variable and the standard relative heart rate as an objective variable, and individual regression equations for every gender and for every age group are stored in the reference storage 36. The relative heart rate denotes, for example, a value indicating relative exercise intensity by a ratio of an actually measured heart rate to a standard maximum heart rate that has been set beforehand for every age. As the standard maximum heart rate for every age, for example, “220−age” may be used. For the elderly, “207−(age×0.7)” may be used.

The standard value derivation unit 37 derives the standard value of the relative heart rate with respect to the measured value of the walking speed that has been acquired, based on the walking heart rate reference corresponding to the gender and the age group of the subject.

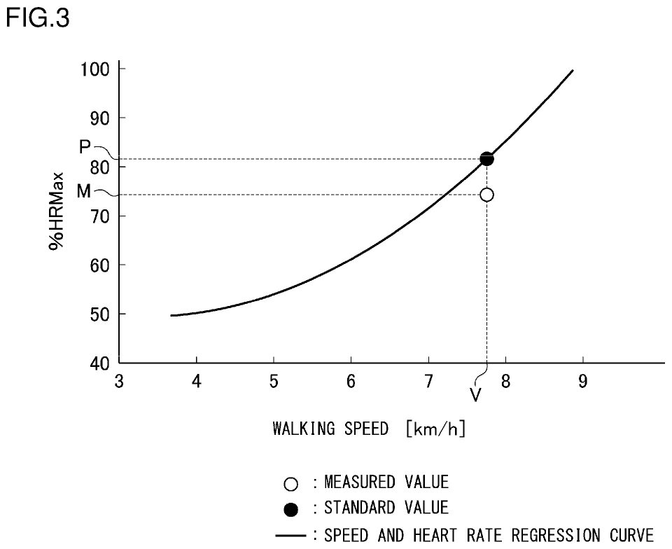

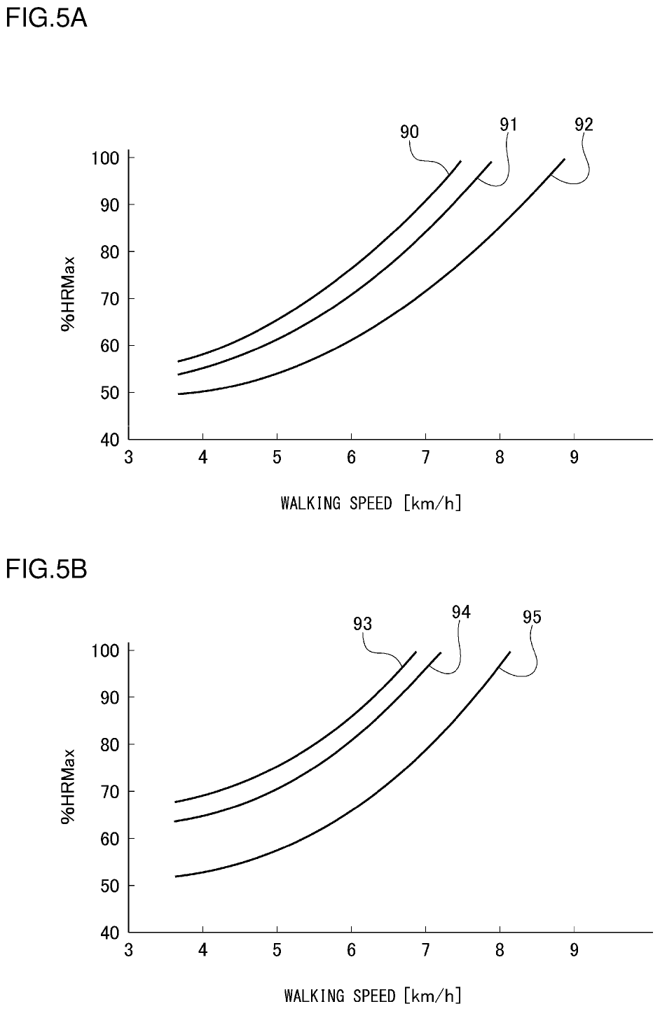

The heart rate score calculation unit 41 calculates a difference between the standard value of the relative heart rate that has been derived by the standard value derivation unit 37 and the measured value of the relative heart rate that has been acquired, and determines a heart rate score based on the difference. The heart rate score calculation unit 41 refers to a measured value in a period of time in which the walking speed is in a high speed range equal to or higher than a predetermined speed, from among the measured values of the heart rate that have been acquired by the heart rate acquisition unit 33. The predetermined speed mentioned here is, for example, a relatively high walking speed, such as seven kilometers per hour. Such a high speed range is a range in which exercise effects are particularly high and the heart rate is also likely to increase moderately. In addition, as illustrated in FIGS. 3, 5A, and 5B, as will be described later, the heart rate tends to increase in a quadratic function, when the walking speed enters a high speed range. Therefore, the heart rate score is determined with reference to the heart rate in the high speed range in which the feature easily appears in particular.

In addition, the heart rate score calculation unit 41 refers to a measured value of the time when the walking time is in a predetermined elapsed period of time, from among the measured values of the heart rate that have been acquired by the heart rate acquisition unit 33. The predetermined elapsed period of time mentioned here is, for example, a period of time from 10 minutes to 30 minutes elapsed from the walking start or the measurement start. The reason why the first 10 minutes are not referred to is that the heart rate is not sufficiently increased at the beginning of walking, and this affects the accuracy. The reason why 30 minutes or later are not referred to is that when the walking time is too long, the heart rate tends to increase due to factors of fatigue and the like, a relatively short walking time is more advantageous, and this affects the accuracy.

The heart rate score calculation unit 41 stores beforehand a table or a regression equation to be referred to for determining the heart rate score from the difference between the standard value and the measured value of the relative heart rate. The measured value of the relative heart rate mentioned here may be an average value of the heart rates in a predetermined speed zone and a predetermined period of time, as described above.

The evaluation value determination unit 42 determines the physical strength level of the subject 10, based on the exercise score that has been determined by the exercise score calculation unit 40 and the heart rate score that has been determined by the heart rate score calculation unit 41. The evaluation value determination unit 42 calculates the physical strength level, by adding a predetermined weight to each the exercise score and the heart rate score and adding them together. For example, an evaluation value of the physical strength level is determined in a calculation formula that physical strength level=exercise score×0.5+heart rate score×0.5. In a case where the exercise score is “80” and the heart rate score is “71”, the physical strength level is 80×0.5+71×0.5=75.5, and is determined to be “76” after rounding off.

As described above, in the present embodiment, in evaluating the physical strength level, the “physical strength level” is evaluated by adding both the “exercise score” in which a result of the effort of the subject 10 such as the length of the walking time and the magnitude of the effect as the walking exercise is taken into consideration and the “heart rate score” in which the potential such as a cardiopulmonary ability of the subject 10 itself is reflected, in an appropriate balance. Note that, in the present embodiment, the description has been given with regard to an example in which the evaluation value of the physical strength level is determined, based on the exercise score and the heart rate score. However, in a modification, the physical strength level may be determined, based on only the heart rate score, in the specifications.

The output unit 46 displays, on a screen, the evaluation value of the physical strength level that has been determined by the evaluation value determination unit 42 and an evaluation comment corresponding to the evaluation value. The output unit 46 stores the evaluation comments for every physical strength level beforehand. The output unit 46 further displays, on the screen, the walking log acquired from the information acquisition unit 39. The output unit 46 may further display the exercise score and the heart rate score together with the evaluation value of the physical strength level. The output unit 46 transmits user data such as the evaluation value of the physical strength level and the walking log to the information management server 50 via the communication unit 44.

The information management server 50 includes a communication unit 51 and a data storage 52. The user data that has been transmitted from the information terminal 30 is stored in the data storage 52 via the communication unit 51 of the information management server 50, and in addition, when a data acquisition request is received from the output unit 46 of the information terminal 30, the user data is read from the data storage 52 and is transmitted to the information terminal 30 via the communication unit 51. The output unit 46 displays, on the screen, the evaluation value of the physical strength level and the walking log, based on the user data that has been received from the information management server 50. In this manner, data of a plurality of subjects is accumulated in the data storage 52 of the information management server 50.

FIG. 3 illustrates a walking heart rate reference in which a correspondence relationship of a standard value of a relative heart rate with respect to a walking speed is defined. In this graph, the horizontal axis represents the walking speed (km/h), and the vertical axis represents the relative heart rate (% HRMax). The relationship of the relative heart rate with respect to the walking speed in the walking exercise is expressed by a quadratic regression curve as illustrated in the drawing. For example, a black circle on the quadratic regression curve indicates that P is the standard value of the relative heart rate with respect to a walking speed V, which has been measured for the subject 10. In contrast, a white circle mark indicates that M was the relative heart rate based on the actually measured heart rate of the subject 10. However, such a white circle mark is not on the quadratic regression curve, and there is a difference on the negative side from the standard value P of the relative heart rate. This indicates that the subject 10 is able to do exercise at a heart rate lower than the standard even at the same walking speed, and the cardiopulmonary ability is higher as the heart rate is lower.

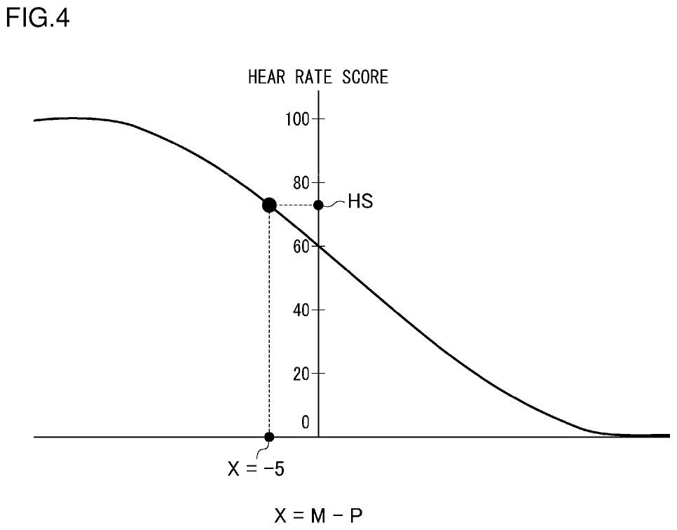

FIG. 4 is a diagram illustrating a relationship between a difference between a standard value and a measured value of the relative heart rate, and a heart rate score. The horizontal axis of the graph in this drawing indicates a difference X (X=measured value of relative heart rate M−standard value P of relative heart rate), and the vertical axis indicates a heart rate score. In the example of FIG. 3, the graph of FIG. 3 indicates that the difference X (measured value M of relative heart rate−standard value P of relative heart rate) is −5, and the heart rate score corresponding to X=−5 is “71”. The smaller the difference X is, the higher the heart rate score is. In particular, the more negative the difference X is, the higher the heart rate score becomes, i.e., “60” or more. The correspondence relationship between the difference X and the heart rate score indicated by this curve is stored beforehand in the form of a table or a regression equation, in the heart rate score calculation unit 41. The correspondence relationship between the difference X and the heart rate score is defined such that the score becomes “60”, in a case where the difference X is 0, that is, in a case where the standard value and the measured value match each other. Note that, in the present embodiment, the description has been given with regard to an example in which the heart rate score is determined, based on the difference between the standard value and the measured value of the relative heart rate. However, in a modification, the heart rate score may be determined, based on the ratio of the measured value to the standard value of the relative heart rate, in the specifications.

FIGS. 5A and 5B illustrate walking heart rate references for every gender and every age group. FIG. 5A illustrates walking heart rate references of males, and FIG. 5B illustrates walking heart rate references of females. The walking heart rate references for every gender and for every age group are stored beforehand in the reference storage 36.

In FIG. 5A, a first reference curve 90 is a quadratic regression curve of the walking heart rate references of males who are 65 years old or older, a second reference curve 91 is a quadratic regression curve of the walking heart rate references of males who are 40 to 64 years old, and a third reference curve 92 is a quadratic regression curve of the walking heart rate references of males who are 20 to 39 years old. As illustrated, the lower the age, the lower the relative heart rate with respect to the same walking speed, and the higher the age, the higher the relative heart rate with respect to the same walking speed.

In FIG. 5B, a fourth reference curve 93 is a quadratic regression curve of the walking heart rate references of females who are 65 years old or older, a fifth reference curve 94 is a quadratic regression curve of the walking heart rate references of females who are 40 to 64 years old, and a sixth reference curve 95 is a quadratic regression curve of the walking heart rate references of females who are 20 to 39 years old. As illustrated, the lower the age, the lower the relative heart rate with respect to the same walking speed, and the higher the age, the higher the relative heart rate with respect to the same walking speed. In addition, the relative heart rate of females with respect to the walking speed tends to be higher than the relative heart rate of males in the same age group illustrated in FIG. 5A. However, as the age of females is lower, the difference in the relative heart rate from males is smaller, and in particular, as the walking speed of females is slower, the difference in the relative heart rate from males is smaller.

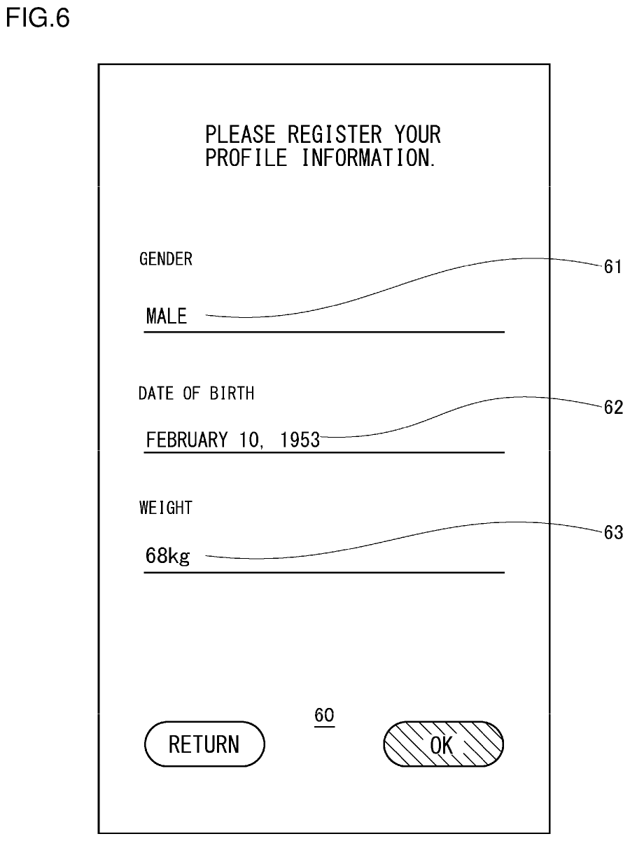

FIG. 6 is a diagram schematically illustrating a screen example for registering attributes of a subject. Prior to the walking exercise, the subject 10 registers its own attribute information beforehand on a walking exercise management application executed on the information terminal 30. On an attribute information registration screen 60, the subject 10 inputs its own gender in a gender field 61, inputs its own date of birth in a date of birth field 62, inputs its own weight in a weight field 63 for registration. Accordingly, the attribute information of the subject 10 is stored in the attribute storage 35.

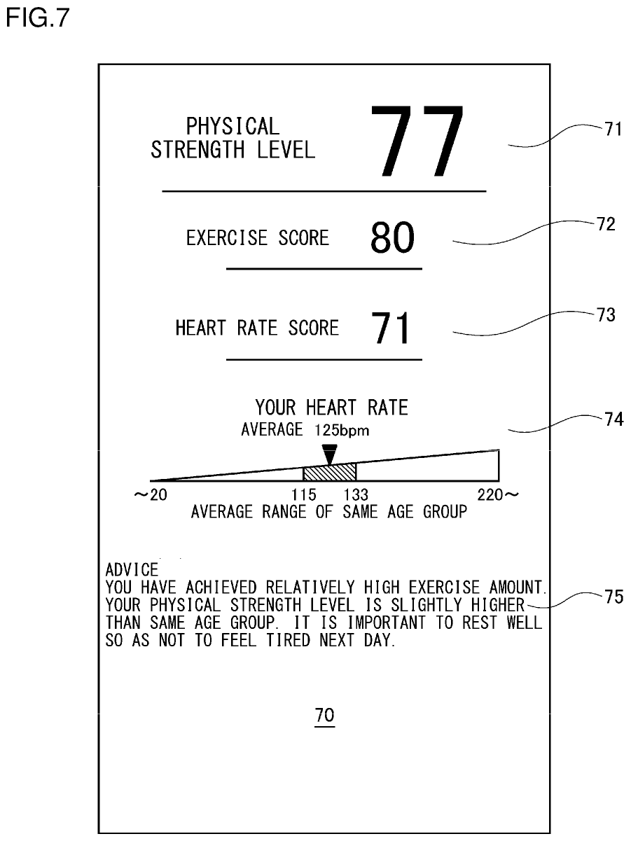

FIG. 7 is a diagram schematically illustrating a screen example of displaying a physical strength level. On a physical strength level display screen 70, “physical strength level 77” is displayed in a physical strength level field 71. In addition, “exercise score 80” is displayed in an exercise score field 72, and “heart rate score 71” is displayed in a heart rate score field 73. In a heart rate field 74, a comparison between the heart rate of the subject 10 and an average heart rate range of the same age group is displayed. In an evaluation comment field 75, an evaluation comment corresponding to the physical strength level is displayed.

### Second Embodiment

The present embodiment is different from the first embodiment in that the evaluation value of the physical strength level is determined by the information management server 50 instead of the information terminal 30, whereas it is determined by the information terminal 30 in the first embodiment. Hereinafter, differences from the first embodiment will be mainly described, and descriptions of common points will be omitted.

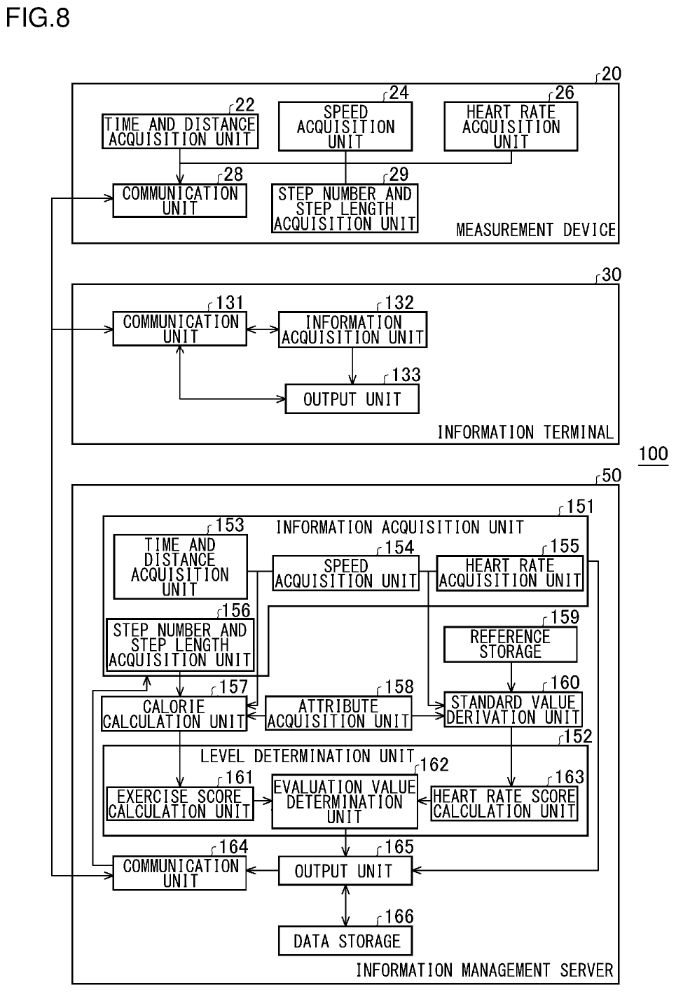

FIG. 8 is a functional block diagram illustrating respective configurations of a physical strength evaluation system 100 in the second embodiment. The measurement device 20 is similar to the measurement device 20 in the first embodiment.

The information terminal 30 in the present embodiment includes a communication unit 131, an information acquisition unit 132, and an output unit 133. The information acquisition unit 132 acquires data of the walking time, the walking distance, the walking speed, the heart rate, the number of steps, and the step length from the measurement device 20 via the communication unit 131, and transmits these data to the information management server 50 via the communication unit 131. The output unit 133 acquires data such as the evaluation value of the physical strength level from the information management server 50 via the communication unit 131, and displays the data on a screen. As described above, the information terminal 30 according to the present embodiment does not perform the process of determining the physical strength level inside the terminal. Instead, the information terminal 30 causes the information management server 50 to perform the process, and displays its results.

The information management server 50 according to the present embodiment has a function of both the information terminal 30 and the information management server 50 in the first embodiment. That is to say, the information management server 50 includes an information acquisition unit 151, a calorie calculation unit 157, an attribute acquisition unit 158, a reference storage 159, a standard value derivation unit 160, a level determination unit 152, a communication unit 164, an output unit 165, and a data storage 166. The information acquisition unit 151, the calorie calculation unit 157, the attribute acquisition unit 158, the reference storage 159, the standard value derivation unit 160, the level determination unit 152, the communication unit 164, and the output unit 165 are functions corresponding to the information acquisition unit 39, the calorie calculation unit 38, the attribute storage 35, the standard value derivation unit 37, the level determination unit 43, the communication unit 44, and the output unit 46 in the first embodiment. The data storage 166 is a function corresponding to the data storage 52 in the first embodiment.

A time and distance acquisition unit 153, a speed acquisition unit 154, a heart rate acquisition unit 155, and a step number and step length acquisition unit 156 included in the information acquisition unit 151 respectively acquire data of the walking time, the walking distance, the walking speed, the heart rate, and the number of steps and the step length from the information terminal 30. The time and distance acquisition unit 153, the speed acquisition unit 154, the heart rate acquisition unit 155, and the step number and step length acquisition unit 156 are functions respectively corresponding to the time and distance acquisition unit 31, the speed acquisition unit 32, the heart rate acquisition unit 33, and the step number and step length acquisition unit 34 in the first embodiment. An exercise score calculation unit 161, a heart rate score calculation unit 162, and an evaluation value determination unit 163 included in the level determination unit 152 are functions respectively corresponding to the exercise score calculation unit 40, the heart rate score calculation unit 41, and the evaluation value determination unit 42 in the first embodiment. As described above, the information terminal 30 in the present embodiment causes the information management server 50 to perform the process of determining the physical strength level and the like, and to return the results to the information terminal 30, and displays the results on the screen of the information terminal 30.

### Third Embodiment

The present embodiment is different from the first and second embodiments in that the characteristic functions in the first and second embodiments are applied to running exercise (so-called running), instead of the walking exercise. Hereinafter, differences from the first embodiment will be mainly described, and descriptions of common points will be omitted. Note that, by replacing parts described as “walking” in the first and second embodiments with “running”, the respective functions are basically applicable to the running exercise.

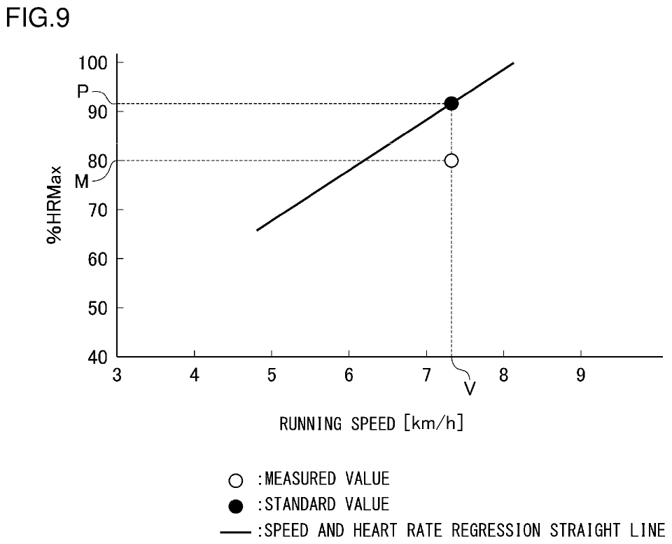

FIG. 9 illustrates a running heart rate reference in which a correspondence relationship of a standard value of a relative heart rate with respect to a running speed is defined. In this graph, the horizontal axis represents the running speed (km/h), and the vertical axis represents the relative heart rate (% HRMax). The relationship of the relative heart rate with respect to the running speed in the running exercise is expressed by a regression straight line as illustrated in the drawing. For example, a black circle on the regression straight line indicates that P is the standard value of the relative heart rate with respect to a running speed V, which has been measured for the subject 10. In contrast, a white circle mark indicates that M was the relative heart rate based on the actually measured heart rate of the subject 10. However, such a white circle mark is not on the regression straight line, and there is a difference on the negative side from the standard value P of the relative heart rate.

As described above, in the running exercise, the relationship between the running speed and the relative heart rate is represented by a regression straight line of a linear expression, and the amount of change in the heart rate with respect to the amount of change in the running speed is constant. These are different from the relationship between the walking speed and the relative heart rate in the walking exercise illustrated in FIG. 3. Therefore, it is necessary to distinguish between the walking exercise and the running exercise and to calculate the heart rate score based on individual heart rate references.

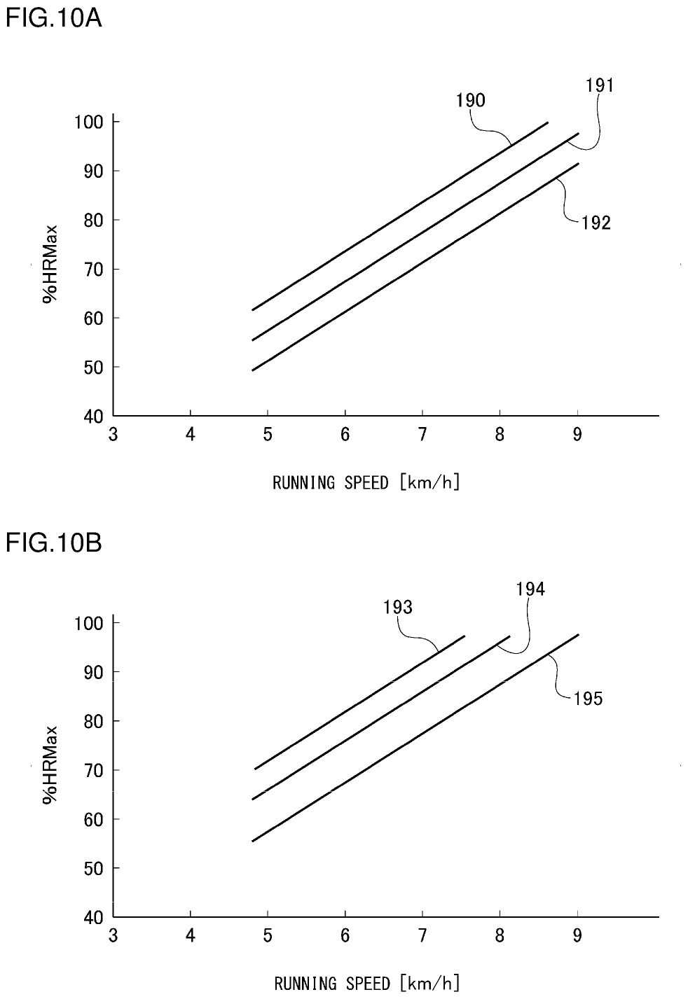

FIGS. 10A and 10B illustrate running heart rate references for every gender and for every age group. FIG. 10A illustrates running heart rate references of males, and FIG. 10B illustrates running heart rate references of females. The running heart rate references for every gender and for every age group are stored beforehand in the reference storage 36.

In FIG. 10A, a first reference straight line 190 is a regression straight line of the running heart rate references of males who are 20 to 39 years old, a second reference straight line 191 is a regression straight line of the running heart rate references of males who are 40 to 64 years old, and a third reference straight line 192 is a regression straight line of the running heart rate references of males who are 65 years old or older. As illustrated, the lower the age, the lower the relative heart rate with respect to the same running speed, and the higher the age, the higher the relative heart rate with respect to the same running speed.

In FIG. 10B, a fourth reference straight line 193 is a regression straight line of the running heart rate references of females who are 20 to 39 years old, a fifth reference straight line 194 is a regression straight line of the running heart rate references of females who are 40 to 64 years old, and a sixth reference straight line 195 is a regression straight line of the running heart rate references of females who are 65 years old or older. As illustrated, the lower the age, the lower the relative heart rate with respect to the same running speed, and the higher the age, the higher the relative heart rate with respect to the same running speed. In addition, the relative heart rate with respect to the running speed of females tends to be higher than the relative heart rate of males in the same age group illustrated in FIG. 10A. However, as the age of females is lower, the difference in the relative heart rate from males is smaller, and in particular, as the running speed of females is slower, the difference in the relative heart rate from males is smaller.

Heretofore, the present invention has been described based on the embodiments. It is to be understood by those skilled in the art that the embodiments are examples, various modifications can be made to combinations of the respective component elements and the respective processing processes, and such modifications are also within the scope of the present invention.

In each of the above embodiments, the description has been given with regard to the aspects in which the information terminal 30 or the information management server 50 evaluates the physical strength level. In a modification, the information terminal 30 may be implemented by being built in the measurement device 20, and the physical strength score may be evaluated in the measurement device 20, in the specifications. In this case, the measurement device 20 transmits the walking log data and the data of the physical strength score to the information terminal 30, and the subject 10 browses the evaluation value of the physical strength score on the screen of the information terminal 30.

In each of the above embodiments, the description has been given with regard to an example of calculating the relative heart rate with the ratio of the measured value of the heart rate to the maximum heart rate in calculation. In a modification, the relative heart rate may be calculated by measuring the heart rate at the time of resting and also calculating the relative heart rate, based on the ratio of the heart rate at the time of doing exercise to the heart rate at the time of resting, or by correcting the ratio to the maximum heart rate using the ratio to the heart rate at the time of resting, in the specifications. In addition, the relative heart rate may be corrected with reference to information indicating external factors such as an outside air temperature, an inclination of a road surface, and an altitude based on the location information.

In each of the above embodiments, the description has been given with regard to the aspects in which the physical strength level is evaluated and displayed in the form of “score”. In a modification, the physical strength level may be evaluated and displayed in the form of “age”, in the specifications. In such a case, for example, depending on which age of the standard value the measured value of the relative heart rate matches, the physical strength level may be expressed like “the physical strength level is 10 years younger than the actual age” by use of the difference between the matched age and the actual age. In this manner, the evaluation value such as “cardiopulmonary age” or “physical strength age” may be used, and in addition, how many ages it is possible to become younger by increasing the number of exercises or extending the exercise time may be presented as an improvement suggestion.

In each of the above embodiments, the description has been given with regard to an example in which fixed values that have been determined beforehand through experiments or sampling are stored in the reference storage 36 or the heart rate score calculation unit 41 for the method for calculating the walking heart rate reference or the heart rate score. In a modification, by analyzing a large number of walking logs that have been collected in the data storage 52 of the information management server 50, a more accurate method for calculating the walking heart rate reference or the heart rate score may be determined and be fed back to the information terminal 30, in the specifications.

In the first and second embodiments, the description has been given with regard to an example of evaluating the physical strength level based on the heart rate in the walking exercise, and in the third embodiment, the description has been given with regard to an example of evaluating the physical strength level based on the heart rate in the running exercise. In a modification, the physical strength level may be evaluated, based on both the walking exercise and the running exercise, or the physical strength level may be evaluated by the subject choosing either the walking exercise or the running exercise, in the specifications.

By generalizing the invention embodied by the above embodiments, the following technical ideas are derived. In order to address the above issues, a physical strength evaluation system according to an aspect of the present invention includes: a reference storage structured to store a walking heart rate reference in which a correspondence relationship of a standard value of a relative heart rate with respect to a walking speed is defined; a speed acquisition unit structured to acquire a measured value of a walking speed of a subject; a heart rate acquisition unit structured to acquire a measured value of a relative heart rate of the subject; a standard value derivation unit structured to derive the standard value of the relative heart rate corresponding to the measured value of the walking speed that has been acquired, based on the walking heart rate reference; a level determination unit structured to determine an evaluation value of a physical strength level of the subject, based on a difference between the measured value of the relative heart rate and the standard value of the relative heart rate; and an output unit structured to output the evaluation value of the physical strength level that has been determined.

Here, the “relative heart rate” may be a ratio of the measured value of the heart rate to the maximum heart rate. The “standard value of the relative heart rate” may be an average value of the relative heart rates that have been measured for a plurality of subjects. According to this aspect, the physical strength level of the subject can be evaluated in a simple method in which the physical strength level is relatively high in a case where the relative heart rate of the subject is lower than the standard value, and the physical strength level is relatively low in a case where the relative heart rate of the subject is higher than the standard value.

The reference storage may store the walking heart rate reference corresponding to an age of a subject, and the standard value derivation unit may derive the standard value of the relative heart rate corresponding to the measured value of the walking speed that has been acquired, based on the walking heart rate reference in accordance with the age of the subject. the reference storage may store the walking heart rate reference corresponding to a gender of a subject, and the standard value derivation unit may derive the standard value of the relative heart rate corresponding to the measured value of the walking speed that has been acquired, based on the walking heart rate reference in accordance with the gender of the subject. Since the walking heart rate reference tends to differ depending on the age or the gender, by storing the walking heart rate references beforehand for every gender and for every age, the physical strength level can be more accurately evaluated, based on a difference from the standard value of the same gender or the same age group of the subject.

The correspondence relationship of the standard value of the relative heart rate with respect to the walking speed defined as the walking heart rate reference may have a relationship in which the standard value of the relative heart rate increases in a quadratic function with respect to an increase in the walking speed, and the level determination unit may determine the evaluation value of the physical strength level of the subject, based on a difference between the measured value of the relative heart rate and the standard value of the relative heart rate in a case where the measured value of the walking speed is in a high speed range equal to or higher than a predetermined speed. The “predetermined speed” may be a relatively fast walking pace such as seven kilometers per hour, for example, and the physical strength level can be evaluated more accurately by measuring the heart rate in a range in which there is an effect on health as walking exercise and the heart rate easily changes.

The speed acquisition unit and the heart rate acquisition unit may acquire the measured value in a predetermined elapsed period of time while the subject is walking, and the level determination unit may determine the evaluation value of the physical strength level of the subject, based on the measured value of the predetermined elapsed period of time. By excluding the heart rate immediately after the start of the walking exercise and the heart rate in the walking exercise that is too long from evaluation targets, the physical strength level can be more accurately evaluated.

The level determination unit may determine the evaluation value of the physical strength level of the subject, based on a difference between the measured value of the relative heart rate and the standard value of the relative heart rate, and a length of a walking time. Here, regarding the “length of walking time”, the physical strength level can be evaluated more accurately by additionally considering whether the length of the walking time has a large effect on health as the walking exercise, that is, by additionally considering a determination factor other than the heart rate. The “length of walking time” may be converted into the calorie consumption by the walking exercise, and may be used as a reference value in the evaluation of the physical strength level.

Another aspect of the present invention is a physical strength evaluation method. This method includes: a process of reading a walking heart rate reference in which a correspondence relationship of a standard value of a relative heart rate with respect to a walking speed is defined; a process of acquiring a measured value of a walking speed of a subject; a process of acquiring a measured value of a relative heart rate of the subject; a process of deriving the standard value of the relative heart rate corresponding to the measured value of the walking speed that has been acquired, based on the walking heart rate reference; a process of determining an evaluation value of a physical strength level of the subject, based on a difference between the measured value of the relative heart rate and the standard value of the relative heart rate; and a process of outputting the evaluation value of the physical strength level that has been determined.

According to this aspect, the physical strength level of the subject can be evaluated in a simple method in which the physical strength level is relatively high in a case where the relative heart rate of the subject is lower than the standard value, and the physical strength level is relatively low in a case where the relative heart rate of the subject is higher than the standard value.

What is claimed is:

1. A physical strength evaluation system comprising:
a reference storage structured to store a walking heart rate reference in which a correspondence relationship of a standard value of a relative heart rate with respect to a walking speed is defined;
a speed acquisition unit structured to acquire a measured value of a walking speed of a subject;
a heart rate acquisition unit structured to acquire a measured value of a relative heart rate of the subject;
a standard value derivation unit structured to derive the standard value of the relative heart rate corresponding to the measured value of the walking speed that has been acquired, based on the walking heart rate reference;
a level determination unit structured to determine an evaluation value of a physical strength level of the subject, based on a difference between the measured value of the relative heart rate and the standard value of the relative heart rate; and
an output unit structured to output the evaluation value of the physical strength level that has been determined.

2. The physical strength evaluation system according to claim 1, wherein
the reference storage stores the walking heart rate reference corresponding to an age of a subject, and
the standard value derivation unit derives the standard value of the relative heart rate corresponding to the measured value of the walking speed that has been acquired, based on the walking heart rate reference in accordance with the age of the subject.

3. The physical strength evaluation system according to claim 1, wherein
the reference storage stores the walking heart rate reference corresponding to a gender of a subject, and
the standard value derivation unit derives the standard value of the relative heart rate corresponding to the measured value of the walking speed that has been acquired, based on the walking heart rate reference in accordance with the gender of the subject.

4. The physical strength evaluation system according to claim 2, wherein
the reference storage stores the walking heart rate reference corresponding to a gender of a subject, and
the standard value derivation unit derives the standard value of the relative heart rate corresponding to the measured value of the walking speed that has been acquired, based on the walking heart rate reference in accordance with the gender of the subject.

5. The physical strength evaluation system according to claim 1, wherein
the correspondence relationship of the standard value of the relative heart rate with respect to the walking speed defined as the walking heart rate reference has a relationship in which the standard value of the relative heart rate increases in a quadratic function with respect to an increase in the walking speed, and
the level determination unit determines the evaluation value of the physical strength level of the subject, based on a difference between the measured value of the relative heart rate and the standard value of the relative heart rate in a case where the measured value of the walking speed is in a high speed range equal to or higher than a predetermined speed.

6. The physical strength evaluation system according to claim 2, wherein
the correspondence relationship of the standard value of the relative heart rate with respect to the walking speed defined as the walking heart rate reference has a relationship in which the standard value of the relative heart rate increases in a quadratic function with respect to an increase in the walking speed, and
the level determination unit determines the evaluation value of the physical strength level of the subject, based on a difference between the measured value of the relative heart rate and the standard value of the relative heart rate in a case where the measured value of the walking speed is in a high speed range equal to or higher than a predetermined speed.

7. The physical strength evaluation system according to claim 3, wherein
the correspondence relationship of the standard value of the relative heart rate with respect to the walking speed defined as the walking heart rate reference has a relationship in which the standard value of the relative heart rate increases in a quadratic function with respect to an increase in the walking speed, and
the level determination unit determines the evaluation value of the physical strength level of the subject, based on a difference between the measured value of the relative heart rate and the standard value of the relative heart rate in a case where the measured value of the walking speed is in a high speed range equal to or higher than a predetermined speed.

8. The physical strength evaluation system according to claim 1, wherein
the speed acquisition unit and the heart rate acquisition unit acquire the measured value in a predetermined elapsed period of time while the subject is walking, and
the level determination unit determines the evaluation value of the physical strength level of the subject, based on the measured value of the predetermined elapsed period of time.

9. The physical strength evaluation system according to claim 2, wherein
the speed acquisition unit and the heart rate acquisition unit acquire the measured value in a predetermined elapsed period of time while the subject is walking, and
the level determination unit determines the evaluation value of the physical strength level of the subject, based on the measured value of the predetermined elapsed period of time.

10. The physical strength evaluation system according to claim 3, wherein
the speed acquisition unit and the heart rate acquisition unit acquire the measured value in a predetermined elapsed period of time while the subject is walking, and
the level determination unit determines the evaluation value of the physical strength level of the subject, based on the measured value of the predetermined elapsed period of time.

11. The physical strength evaluation system according to claim 1, wherein the level determination unit determines the evaluation value of the physical strength level of the subject, based on a difference between the measured value of the relative heart rate and the standard value of the relative heart rate, and a length of a walking time.

12. The physical strength evaluation system according to claim 2, wherein the level determination unit determines the evaluation value of the physical strength level of the subject, based on a difference between the measured value of the relative heart rate and the standard value of the relative heart rate, and a length of a walking time.

13. The physical strength evaluation system according to claim 3, wherein the level determination unit determines the evaluation value of the physical strength level of the subject, based on a difference between the measured value of the relative heart rate and the standard value of the relative heart rate, and a length of a walking time.

14. A physical strength evaluation method comprising:
a process of reading a walking heart rate reference in which a correspondence relationship of a standard value of a relative heart rate with respect to a walking speed is defined;
a process of acquiring a measured value of a walking speed of a subject;
a process of acquiring a measured value of a relative heart rate of the subject;
a process of deriving the standard value of the relative heart rate corresponding to the measured value of the walking speed that has been acquired, based on the walking heart rate reference;
a process of determining an evaluation value of a physical strength level of the subject, based on a difference between the measured value of the relative heart rate and the standard value of the relative heart rate; and
a process of outputting the evaluation value of the physical strength level that has been determined.

15. A non-transitory computer-readable storage medium storing a program for causing a computer to execute processing comprising:
a function of storing a walking heart rate reference in which a correspondence relationship of a standard value of a relative heart rate with respect to a walking speed is defined;
a function of acquiring a measured value of a walking speed of a subject;
a function of acquiring a measured value of a relative heart rate of the subject;
a function of deriving the standard value of the relative heart rate corresponding to the measured value of the walking speed that has been acquired, based on the walking heart rate reference;
a function of determining an evaluation value of a physical strength level of the subject, based on a difference between the measured value of the relative heart rate and the standard value of the relative heart rate; and
a function of outputting the evaluation value of the physical strength level that has been determined.
